(19) United States
(12) Patent Application Publication
(10) Pub. No.: US 2020/0167658 A1
(43) Pub. Date: May 28, 2020

(54) System of Portable Real Time Neurofeedback Training

(71) Applicant: Jessica Du, Jackson, NJ (US)

(72) Inventor: Jessica Du, Jackson, NJ (US)

(21) Appl. No.: 16/199,141

(22) Filed: Nov. 24, 2018

(51) Int. Cl. G06N 3/08; G06N 3/04; A61B 5/00; A61B 5/0476; A61B 5/04; A61M 21/02

(52) U.S. Cl. CPC G06N 3/088; G06N 3/0445; G06N 3/04; G06N 3/0454; A61B 5/0006; A61M 2021/0027; A61B 5/7267; A61B 5/04012; A61M 21/02; A61M 2205/8206; A61B 5/0476

(57) ABSTRACT

A first device collects human user brainwave data and transfers it to a second device through Bluetooth/USB. The second device uses artificial intelligence to process the data received from the first device then ports trained Deep Learning models to the third device. Human users use the third device which provides neurofeedback services to change the current brainwave state to the desired state.

### TECHNICAL FIELD

This specification relates to systems and methods for collecting and managing brainwave data, more particularly to systems and methods for providing real time neurofeedback training services.

### BACKGROUND

In today's world, more and more people need to complete an increasing amount of tasks in a very limited time frame. To be able to do so is becoming a challenge as changing from a state of concentration to relaxation and vice versa with high efficiency is critical to have effective results. Helping people to achieve such goal is important and desirable.

Today many people use smartphones, making it for them to change their brainwave states at any place and time by leveraging artificial intelligence and adding neurofeedback services.

### SUMMARY

In accordance with an embodiment, a method of obtaining and processing information relating to data in a system is provided. The system includes a wearable, a deep learning training system, a human user and a smart phone. A first device in the system, having EEG sensors, receives brainwave data from human user, stored and processed, then transmit to second device or third device. In one embodiment, the system uses USB/Bluetooth for communication. In one embodiment, the EEG sensor in the first device receives brainwave data, stores, compresses, and transmits to the second or third device through USB or Bluetooth. In one embodiment, the second device comprises deep learning training hardware such as GPU, memory, CPU, software such as Deep learning implementations. In one embodiment, the third device comprises GPU, memory (storage), trained Deep Learning model implementations, and neurofeedback services.

In accordance with another embodiment, a method of processing information relating to data from the system is provided. The second device receives data from first device and feed to deep learning training system. In one embodiment, the deep learning training system implements models such as RNN (Recurrent Neural Network), CNN (Convolutional Neural Network), GAN (Generative Adversarial Network) and LSTM (Long Short Time Memory), those models are trained with different type of brainwave data. Those Deep Learning models are used to provide key features in neurofeedback. In one embodiment, RNN uses changing brainwave recommendation from one to another, feature learning to enhance collaborative filtering. In one embodiment, RNN uses neural style transfer to generate desired pictures. In one embodiment, desired video is to be generated for desired brain state. In one embodiment, use CNN to extract features from brainwave signals, the content features could be used to cluster similar signals to produce personalized neural style transfer.

In accordance with another embodiment, a method of providing neurofeedback service is provided. With desired state of neurofeedback, real time brainwave data is collected from device 1 and transmitted to device 2, where neurofeedback service management generates arts pictures or video for human user to watch. Neurofeedback service management generates new arts pictures or videos to lead human brainwaves to desired state.

### BRIEF DESCRIPTION OF THE DRAWINGS

FIG. 1, shows a neurofeedback system that may be used to provide deep learning and neurofeedback services in accordance with an embodiment.

FIG. 2 show components of wearable device in accordance with an embodiment.

FIG. 3 show s functional components of a deep learning system in accordance with an embodiment.

FIG. 4 shows functional components of a portable neurofeedback device in accordance with embodiment.

FIG. 5 shows functional components of a deep CNN model which plugs in visual feature into content images during neural style transfer process in accordance with an embodiment.

FIG. 6 shows functional components of feature extraction from pretrained deep CNN along with content images to produce brainwave influential images.

FIG. 7 shows functional components of real time neurofeedback training process with and embodiment.

### DETAILED DESCRIPTION

In accordance with various embodiments, methods and systems for providing neurofeedback services and Deep Learning management services. In accordance with embodiments described herein, a wearable device is used. The wearable device obtains information of brainwaves then another device uses the information obtained to train DL models or identify the state of brain, this information is to be used for desired brainwave state training.

In accordance with one embodiment, a wearable device has sensors to collect brainwave information in multiple channels, it converts analog data to digital data, it stores data locally and also transmit data to another device through Bluetooth. The data transfer destination could be a computer or a portable device such as a smart phone.

In accordance with one embodiment, a Deep Learning training system is a computer, which has CPU, GPU, memory, Bluetooth and USB hardware components, it also has implementation of Deep Learning algorithms such as CNN, RNN, GAN and LSTM etc, trained with brainwave Delta wave data, Theta wave data, Alpha wave data and Beta wave data, with ability of DL transfer learning.

In another embodiment, a pretrained deep CNN is used to extract specific visual feature and uses neural style transfer to generate desired images.

In another embodiment, a program is used to generate videos from pictures.

In another embodiment, a portable device such as a smart phone has GPU, memory and wireless enable controller which provide Bluetooth capability. It has DL models trained and ported over from DLTS. The device receives real time brainwave data for neurofeedback process.

The methods, systems and apparatus described herein allow a mobile neurofeedback system to be used at anytime and anywhere for neurofeedback services.

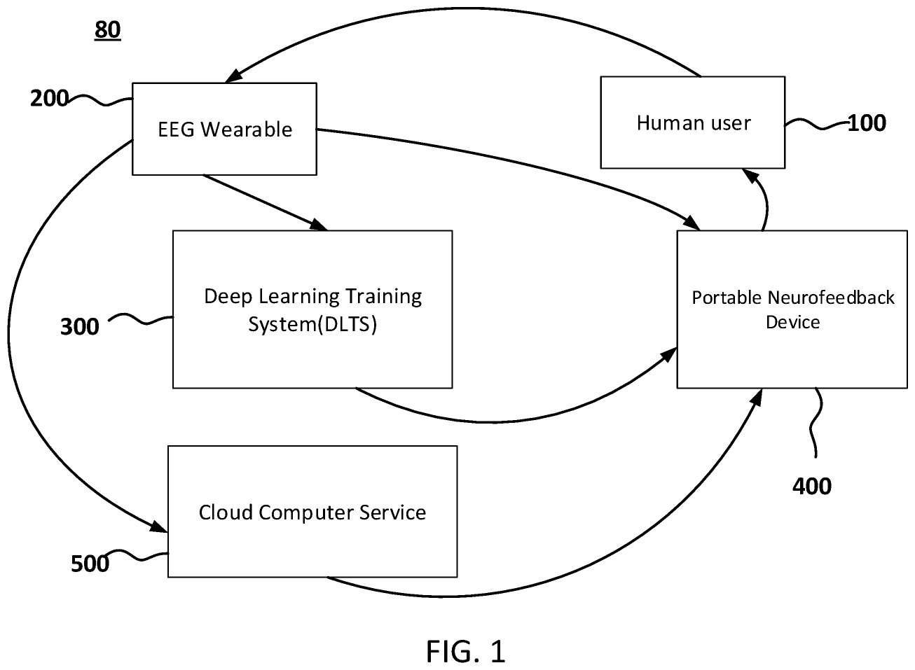

FIG. 1 shows a system 80 that may be used to provide data collection and neurofeedback services in accordance with an embodiment. System 80 includes a human user 100, a wearable device 200, a Deep Learning training system 300, a Cloud computer service 500 and a portable device 400.

Wearable device 200 collects data. For example, it may collect multiple type of sensor data, including, without limitation, brainwave data, step counts, quality of sleep, distance traveled, sleep time, heart rate, calories burned, deep sleep, eating habits etc. Wearable device 200 may from time to time receive specified data from human user 100 and store the specific data. Wearable device 200 may send data to another system such as system 300 and/or system 500, and/or system 400. Wearable device 200 communicates with another system through USB or Bluetooth/WiFi.

System 300 from time to time request data from system 200 and communicate with system 200 to retrieve the requested data. System 300 is connected to system 200 through USB/Bluetooth/WiFi, and may be a personal computer, a server etc. In some embodiments, system 200 may be a cluster of servers.

System 400 from time to time requests data from system 200 and communicates with system 200 to retrieve the requested data. System 400 is connected to system 200 through Bluetooth. For example, system 400 may be a smart phone.

System 500 periodically or in real time requests data from system 200 and communicates with system 200 to retrieve the requested data. System 500 communicates with system 400 through wireless/WiFi.

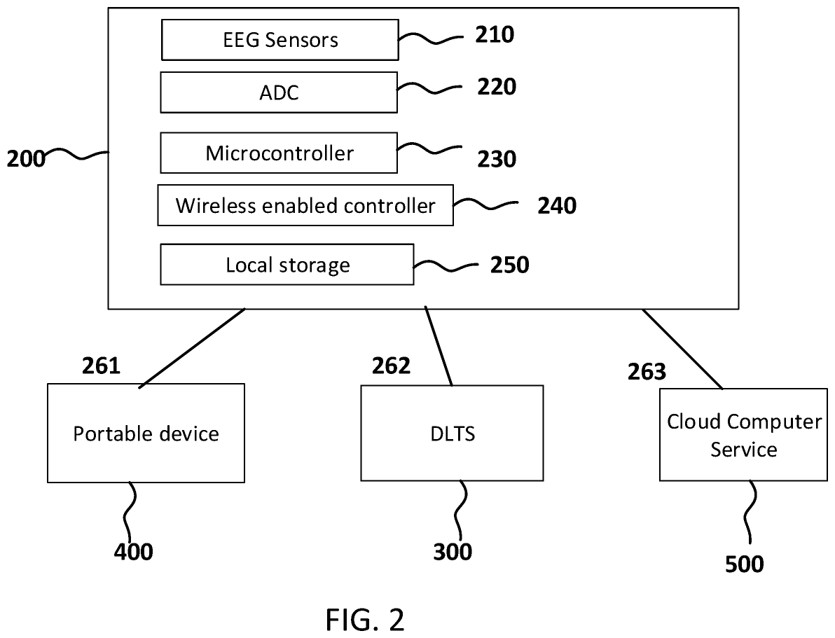

FIG. 2 shows components of wearable device 200 in accordance with an embodiment. Wearable device 200 includes EEG sensors 210, an Analog-to-Digital converter (ADC) 220, a microcontroller 230, wireless enabled controller 240 and local storage 250.

In the illustrative embodiment, link 261 connects wearable device 200 to portable device 400 via Bluetooth. Link 262 connects wearable device 200 to DLTS system 300 via USB. Link 263 connects wearable device 200 to cloud 500 via wireless carriers/WiFi.

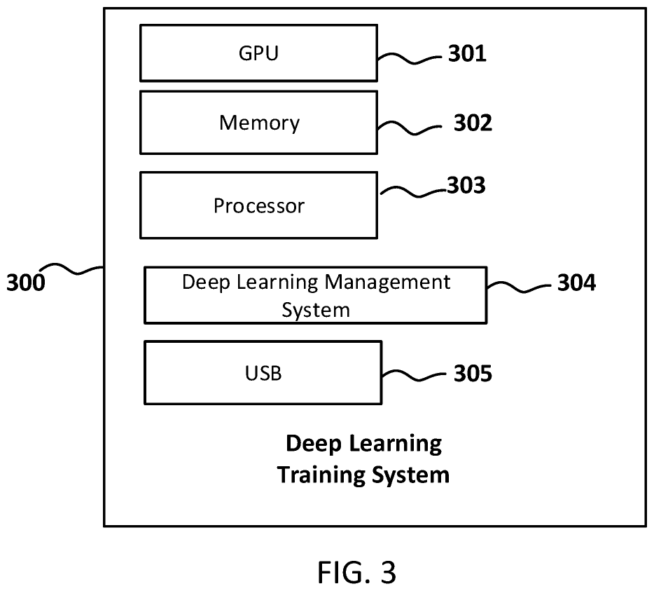

FIG. 3 shows functional components of Deep Learning training system 300 in accordance with an embodiment. FIG. 3 and the discussion below is equally applicable to any computers in cloud computer service 500. DLTS 300 includes GPU 301, memory 302, CPU 303, USB 305 and deep learning management system 304.

Deep Learning management system 304 controls the activities of various components within DLTS 300. Deep Learning management system 304 includes implementation of various DL models such as CNN, RNN, LSTM and GAN etc, as well as trained models with different type of brainwave data relating to delta wave, theta wave, alpha wave and beta wave data from wearable device 200. RNN uses neural style transfer to generate desired pictures and videos for desired brain state.

DLTS 300 has Deep Learning management system 304 which implements pretrained CNN on ImageNet. It has multiple Cony layers and fully connected layers.

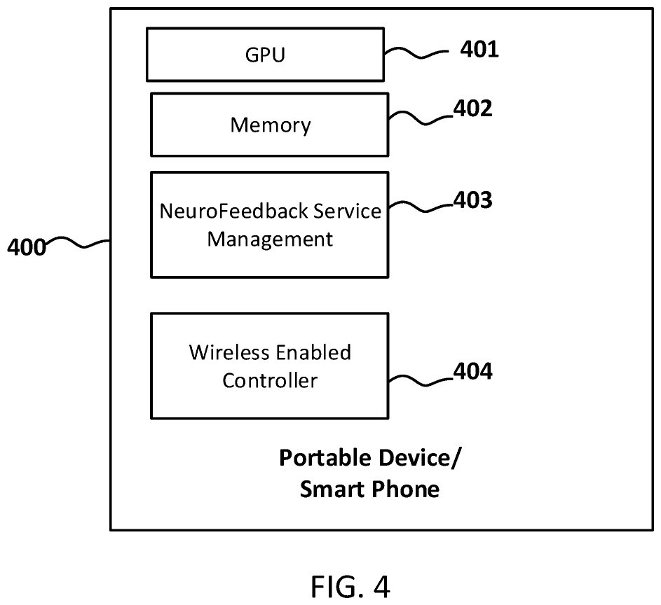

FIG. 4 shows functional components of portable device 400 in accordance with an embodiment. Portable device 400 includes GPU 401, memory 402, neurofeedback management service and wireless enabled controller 404. Neurofeedback management service 403 controls the operations of various components of portable device 400. Neurofeedback management service 403 includes trained deep learning models ported over from DLTS 300 or 500, it manages brainwave data transmitted from wearable device 200, visualizes brain activities in real time, and manages desired brainwave state with deep learning recommendations through watching real time generated videos from deep learning neuro style transfer process.

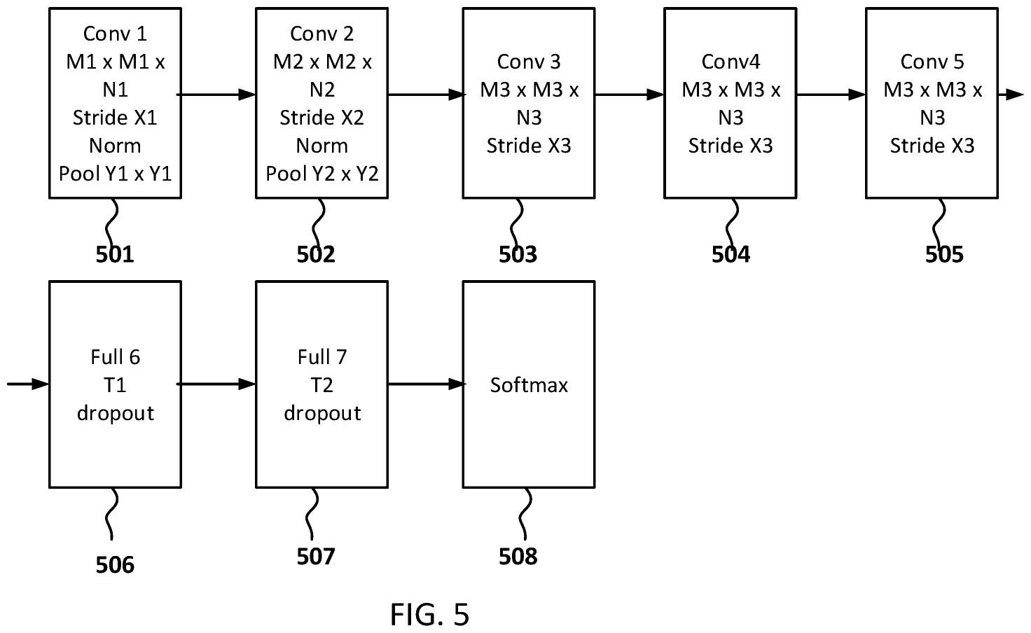

FIG. 5 shows functional components of an example of CNN model. Consolidated Cony layer 501 has RGB image of M1×M1×N1 with stride X1 normalization, pooling of Y1×Y1, Cony layer 502 has RGB image of M2×M2×N2 with stride X2 normalization, pooling of Y2×Y2, Cony layer 503 has RGB image of M3×M3×N3 with stride X3, Cony layer 504 has RGB image of M3×M3×N3 with stride X3, Cony layer 505 has RGB image of M3×M3×N3 with stride X3, fully connected layer 506 has T1 dropout, fully connected layer 507 has T2 dropout, Softmax layer 508 is used to produce multiple outputs.

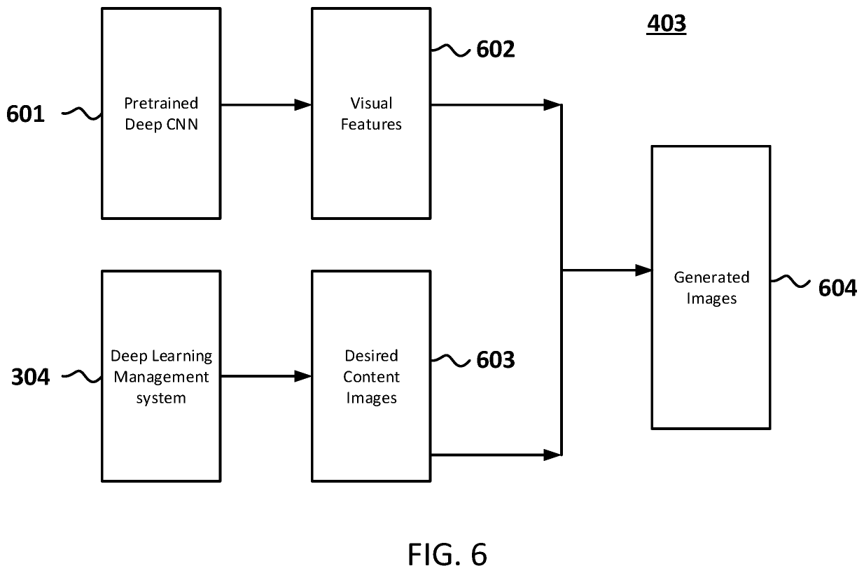

FIG. 6 shows one of neurofeedback service management 403 is functional components, Pretrained Deep CNN model 601 extracts specific visual features 602 requested by human user, based on recommendation from Deep Learning management system 304 the corresponding content images 603 are used to generate desired images 604.

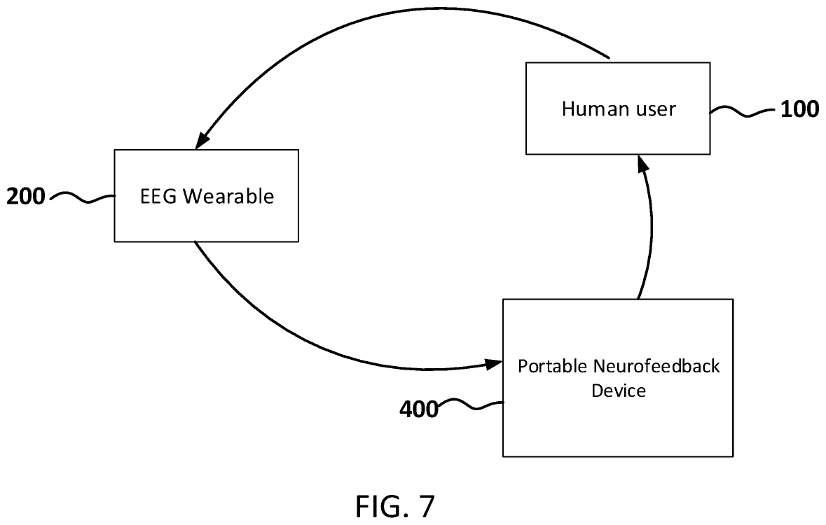

FIG. 7 shows device 200 collects real time brain wave data from human user 100 and transmits to device 400, neurofeedback service management system generates arts pictures/videos for human user to watch. Corresponding brave wave data of reflection is collected from device 200 transmits to device 400, neurofeedback service management system generates new arts pictures/videos to lead brainwave to desired state.

### CITATION LIST

PATENT CITATIONS

| Publication Number | Priority date | Publication date | Assignee | Title |
|---|---|---|---|---|
| U.S. Pat. No. 9,263,036B1 | 2012 Nov. 29 | 2016 Feb. 16 | Google Inc | System and method for speech recognition |
| | | | | using deep recurrent neural networks |
| US20160099010A1 | 2014 Oct. 3 | 2016 Apr. 7 | Google Inc | Convolutional, long short-term memory, |
| | | | | fully connected deep neural networks |
| US20140270488A1 | 2013 Mar. 14 | 2014 Oct. 28 | Google Inc | Method and apparatus for |
| | | | | characterizing an image |
| U.S. Pat. No. 4,736,751A | 1986 Dec. 16 | 1988 Apr. 12 | EEG Systems Labs | Brain wave source network |
| | | | | location scanning method and system |
| U.S. Pat. No. 5,899,867A | 1996 Oct. 11 | 1999 May 4 | Thomas F. Collura | System for self-administration |
| | | | | of electroencephalographic (EEG) |
| | | | | neurofeedback training |

### Non-Patent Citations

Very deep convolutional networks for large-scale image recognition, Karen Simonyan & Andrew Zisserman.

Going Deeper with Convolutions, Christian Szegedy, Wei Liu, Yangqing Jia, Pierre Sermanet, Scott Reed, Dragomir Anguelov, Dumitru Erhan, Vincent Vanhoucke, Andrew Rabinovich.

ImageNet Classification with Deep Convolutional Neural Networks, Alex Krizhevsky, Ilya Sutskever, Geoffrey E. Hinton

A Neural Algorithm of Artistic Style, Leon A. Gatys, Alexander S. Ecker, Matthias Bethge.

Conditional Image Generation with PixelCNN Decoders, Aaron van den Oord, Nal Kalchbrenner, Oriol Vinyals, Lasse Espeholt, Alex Graves, Koray Kavukcuoglu.

Deep Visual-Semantic Alignments for Generating Image Descriptions, Andrej Karpathy, Li Fei-Fei.

1. A method of collecting sensor data from human user, the method comprising:
Receiving brainwaves from human user by a device attached/implanted to the user. The sensor data is stored locally on the device and transmitted to another system for further processing.

2. The method of claim 1, wherein the device is a wearable device.

3. The method of claim 1, wherein the information transferred to another system is through wireless with encrypted channels or through USB.

4. A method of brainwave training through Deep Learning, the method comprising:
Deep Learning algorithms; a music library and a picture library.

5. The method of claim 4, wherein the Deep Learning algorithms are Recurrent Neural Network (RNN), Long short time memory (LSTM), Convolutional Neural Network (CNN), Generative adversarial Network (GAN), or variants such as Gated Recurrent Unit (GRU) or combination of RNN, LSTM, CNN, GAN.

6. A method of sound and video generation and real time processing:
The method comprising:
Registering wearable devices and authenticate, receiving information from wearable devices through secure channels.
Activating desired brainwave entrainment functions.
Generating desired sound and video based on adaptive learning goal and data received from wearable, and further tuning deep learning models.
Adjusting corresponding sound and visual entrainment functions.

7. The method of claim 6, wherein the Deep Learning models are trained RNN, LSTM, CNN, GAN, GRU, mixed RNN, LSTM, CNN, GAN, GRU models.

8. The method of claim 6, where the brainwave entrainment functions make up a training program to lead brainwave to desired state.

9. A device attached to human body, the device comprising:
An EEG sensor;
A rechargeable battery;
A memory storing computer program instructions;
Multiple processors configured to execute computer program which processors to perform operations comprising:
Collecting and processing data from sensor.
Transmitting data to another system;
A wireless communication solution (WiFi/Bluetooth)

10. The device of claim 9, wherein the registration procedure comprises registration by biometric information.

11. The device of claim 9, wherein the operations further comprises:
Collecting raw data, filling and sending it to another system for processing.

12. The device of claim 9, wherein wireless communication solution comprises ultra-low-power radio solution or other type.

13. Deep learning training system device; the device comprising:
A memory storing computer program instruction;
A processor configured to execute computer program instructions;
A GPU configured for high parallel computation tasks;
Recurrent neural network (RNN) algorithm implementations;
Long short time memory (LSTM) algorithm implementations;
Generative Adversarial network (GAN) algorithm implementations;
Convolutional neural network algorithm implementations;
A music/sound track library which is used to train different Deep Learning models for stress reduction, relaxation, sleep enhancement, mega-learning, peak-performance, meditation or high state of consciousness.

14. The device of claim 13, the operations further comprising:
Receiving sensor data from wearable device as input data of different Deep Learning models.

15. The device of claim 13, the operations further comprising:
Feature learning to enhance collaborative filtering in CNN.

16. The device of claim 13, the operations further comprising:
Providing recommendations based on desired brainwave state.

17. The device of claim 13, the operation further comprising:
Generating art pictures through new style transfer.

18. The device of claim 13, the operation further comprising:
Generating videos suitable for desired brainwave states.

19. A device used by user (human), the device comprising:
A memory storing computer program instructions;
A processor configured to execute the computer program instruction which, when executed on the processor, cause the processor to perform operations comprising:
Identifying wearable device ID in a registration procedure;
Receiving sensor data from wearable device in real time;
Receiving instructions from device user for desired brainwave state;
Processing received sensor data and deliver audio-visual brainwave entrainment.
Measuring and storing brainwave state changes against targeted goals;
Recommending actions in comparison to ideal brainwave state to be achieved.

20. The device of claim 19, wherein the operations further comprises:
Adjusting Deep Learning algorithm parameters to generate new video and sound for human user to use.
